(19) United States
(12) Patent Application Publication
(10) Pub. No.: US 2005/0032782 A1
(43) Pub. Date: Feb. 10, 2005

(54) Treatment of chronic pain associated with drug or radiation therapy

(75) Inventors: Srinivas G. Rao, Encinitas, CA (US); Jay D. Kranzler, La Jolla, CA (US)

Correspondence Address: DARBY & DARBY P.C., P. O. BOX 5257, NEW YORK, NY, 10150-5257, US

(73) Assignee: Cypress Bioscience, Inc.

(21) Appl. No.: 10/850,939

(22) Filed: May 21, 2004

Related U.S. Application Data
(60) Provisional application No. 60/473,377, filed on May 23, 2003.

(51) Int. Cl. A61K 31/55; A61K 31/137

(52) U.S. Cl. 514/220; 514/650

(57) ABSTRACT

Methods for treating chronic widespread pain associated with drug therapy or radiation therapy are described. The method generally involves administering a therapeutically effective amount of a dual or tri reuptake inhibitor of a specific type or a pharmaceutically acceptable salt thereof. Preferably the compound is a non-tricyclic dual reuptake inhibitor. The most preferred compound is milnacipran or a bioequivalent or pharmaceutically acceptable salt thereof. Other preferred compounds are duloxetine and venlafaxine or a bioequivalent or pharmaceutically acceptable salt thereof. In yet another embodiment, a therapeutically effective amount of a non-tricyclic triple reuptake inhibitor (“TRI”) compound of a specific type, or a pharmaceutically acceptable salt thereof, is administered. The TRI compounds are characterized by their ability to block the reuptake (and, hence, increase central concentrations of) the three primary brain monoamines: serotonin, noradrenaline, and dopamine.

### CROSS-REFERENCE TO RELATED APPLICATIONS

This application claims the benefit of U.S. Provisional Application No. 60/473,377 filed in the United States Patent and Trademark Office on May 23, 2003.

### FIELD OF THE INVENTION

The present invention is in the field of treating chronic pain that is associated with radiation or drug therapy. Most preferably in the field of treating chronic pain arising from drug or radiation therapy to treat cancer.

### BACKGROUND OF THE INVENTION

Drug or radiation therapy is commonly used to treat illnesses such as cancer, rheumatoid arthritis, autoimmune disease, and viral infections. While these approaches are presently the most effective means of treatment, they are not without sometimes very harsh side effects. Cancer, for example, is diagnosed in over one million Americans each year. Approximately 8 million Americans either currently have cancer or have a history of cancer (Jacox et al 1994 Management of Cancer Pain. Clinical Practice Guideline No. 9. AHCPR Publication No. 94-0592, U.S. Dept of Health and Human Services, Rockville, Md.). Current therapies to treat cancer include radiation therapy, chemotherapy and surgery, While these therapies are the most effective, they are not without side effects. Side effects of cancer treatment can include nausea, fatigue and chronic pain. For example, chronic pain syndromes following breast cancer treatment has been estimated to occur in 20-25% patients undergoing axillary (armpit) dissection, with or without mastectomy, and appears to correlate with the extent of axillary surgery. Polyneuropathies can be caused by chemotherapy and radiation therapy. Radiation therapy can contribute to the development of chronic pain in patients treated for breast, prostate and Hodgkin's lymphoma (Tasmuth et al 1997 Acta Oncol 36(6):625-30; McFarlane et al. 2002 Clin Oncol 14(6):468-471; Antolak et al 2002 J Urol 167(6):2525). Patients may also develop chronic widespread pain induced by premature ovarian failure/premature menopause induced by chemotherapy and other drugs used to treat the cancer.

Often the side effects of chemotherapy limit the use of these drugs for treatment. The most common side effects are bone marrow suppression, neutropenia, renal toxicity and the induction of peripheral neuropathy. These often result in termination of treatment or alteration of the dose. The type of resulting neuropathy is dependent on the type of therapeutic substance used. Platinum derivatives such as cisplatin, oxaliplatin and carboplatin result in a pure sensory and painful neuropathy while substances like vinscristine, taxol and suramin cause a mixed sensorimotor neuropathy with or without involvement of the autonomic nervous system.

The neurotoxicity caused by the chemotherapy is dependant on the total cumulative dose, duration of treatment and type of substance used. In some instances, neuropathy can develop after a single drug application although it is known that neurotoxicity can occur immediately during or shortly after drug administration. Neurotoxic effects can become evident a long time after the end of the treatment. This is referred to as “coasting”. In general, the peripheral nervous system is capable of regeneration after injury if the cell body is spared and no further damage occurs during the repair period. However, in some situations, chemotherapy-induced neuropathy is only partly reversible and in the worst case damage is completely irreversible.

Little is known about the mechanisms responsible for development of neuropathy. Most of the studies to date have focused on changes in tissue morphology with treatment. Paclitaxel-mediated sensory neuropathy is thought to be due to an axonopathy, dorsal root ganglionopathy, Schwann cell abnormality or a combination thereof which resolves slowly (Rowinski et al 1993 J Natl Cancer Inst 15:107-115; Chaudhry et al 1994 Ann Neurol 35:304-311; Lipton et al 1989 Neurology 39:368-373; Forsyth et al 1997 J Neurooncol 35:47-53). Risk factors for developing neuropathy after chemotherapy include previous nerve damage from diabetes, alcohol use/abuse or inherited neuropathy.

To date there is no effective strategy to prevent or cure the symptoms of chemotherapy-induced neuropathy. Therapy is restricted to the treatment of unpleasant dysaesthesia and pain by using membrane stabilizing drugs and tricyclic antidepressants (TCAs). TCAs block the reuptake of serotonin and noradrenaline and serve as a first-line treatment of neuropathic pain (Kvinesdale et al 1984 J Am Med Ass 45:47-52; Bowsher 1991 Br Med Bull 47:644-646; Lynch 2001 J Psychiat Neurosci 26:30-36; Egbunike and Chaffe 1990 Pharmacotherapy 10:262-270). Tricyclic antidepressants are a well-recognized class of antidepressant compounds and are characterized by a fused tricyclic nucleus. TCAs have previously been shown to provide modest analgesia for neuropathic cancer pain but have numerous side effects (Hammack et al Pain 2002 98:195-203, Farrar and Portenoy Oncol 2001 15:1435-1442, Ehrnrooth et al Acta Oncol 2001 40:745-750). These are not preferred for use as described herein. Side effects of TCA administration include anticholinergic reactions (e.g. dry mouth), cognitive effects, hypotension, cardiac arrythmia, urinary retention and somnolence. Compounds that are commonly classified as tricyclic antidepressants include imipramine, desipramine, clomipramine, trimipramine, amitriptyline, nortriptyline, doxepin, and protriptyline. The use of these agents is limited by their numerous side effects even at low doses, rendering them less desirable as therapy.

Selective serotonin reuptake inhibitor antidepressants have been found to be less effective for neuropathic pain (Sindrup and Jensen 1999 Pain 83:389-300; Galer 1995 Neurology 45(suppl 9):S17-S25; Calissi and Jaber 1995 Ann Pharmacother 29:769-777).

Nerve growth factor failed in a phase III trial for treatment of painful diabetic. Novel growth factor therapies such as administration of glia-derived neurotrophic factor (GDNF) for analgesia have not yet reached clinical application (Boucher et al 2000 Science 290: 124-127). This is due to reasons such as difficulties in drug administration, adverse effects and pharmacokinetics. No treatment has demonstrated activity for the treatment of severe paclitaxel-induced neuropathies.

It is therefore an object of the present invention to provide a method of treatment for widespread chronic pain associated with drug or radiation therapy.

### SUMMARY OF THE INVENTION

Methods for treating chronic widespread pain associated with drug therapy or radiation therapy are described. The method generally involves administering a therapeutically effective amount of a monoamine reuptake inhibitor of a specific type or a pharmaceutically acceptable salt thereof. Preferably the compound is a dual reuptake inhibitor (“DRI”) which is not a tricyclic serotonin-norepinephrine reuptake inhibitor (“SNRI”). Either DRIs where serotonin reuptake inhibition is greater than norepinephrine reuptake inhibition, or where norepinephrine reuptake inhibition is greater than serotonin reuptake inhibition may be used. The most preferred compound is milnacipran or a bioequivalent or pharmaceutically acceptable salt thereof. Other preferred compounds are duloxetine and venlafaxine or a bioequivalent or pharmaceutically acceptable salt thereof. Alternatively, a therapeutically effective amount of a non-tricyclic triple reuptake inhibitor (“TRI”) compound of a specific type, or a pharmaceutically acceptable salt thereof, is administered.

These compounds are administered to a patient in need of treatment thereof at the time of treatment or following treatment, as needed in an amount effective to reduce pain due to the chemotherapy or radiation.

### DETAILED DESCRIPTION OF THE INVENTION

Abbreviations

| | |
|---|---|
| 5-HT | serotonin |
| NE | norepinephrine (noradrenaline) |
| DA | dopamine |
| NMDA | N-methyl D-aspartate |
| NSAIDs | non-steroidal anti-inflammatory drugs |
| SSRIs | selective serotonin reuptake inhibitors |
| TCAs | tricycic antidepressants |
| DRI | dual reuptake inhibitors, a class of compounds that block |
| | the reuptake of 5-HT and NE. This class can be further |
| | broken into SNRI and NSRI subclasses. |
| SNRIs | dual serotonin norepinephrine reuptake inhibitors, |
| | where werotonin reuptake exceeds norepinephrine |
| | reuptake, 5-HT > NE. |
| NSRI | dual norepinephrine reuptake inhibitor where |
| | norepinephrine reuptake exceeds serotonin reuptake, |
| | NE > 5-HT DRI. |
| TRI | a compound that blocks the reuptake of 5-HT, NE, and |
| | DA |

Definitions

The term “dual serotonin norepinephrine reuptake inhibitor compound” (also referred herein as DRI compounds) refers to compounds that inhibit reuptake of serotonin and norepinephrine.

The term “NSRI” refers to a particular subclass of DRI compounds that inhibit the reuptake of norepinephrine more than they inhibit reuptake of serotonin. The term SNRI refers to DRI compounds that inhibit the reuptake of serotonin more than they inhibit reuptake of norepinephrine.

The term TRI refers to a class of compounds with antidepressant, anorectic, and anti-Parkinsonian properties that inhibit the reuptake of serotonin, noradrenaline, and dopamine.

I. Chronic Pain Conditions to be Treated

Drug or radiation treatment is used in treating cancers such as bone cancer, brain cancer, breast cancer, endocrine system cancer, gastrointestinal cancer, ovarian cancer, head and neck cancer, leukemia, lung cancer, lymphoma, myeloma, prostate cancer, sarcoma, skin cancer, urogenital cancer and thyroid cancer. Chemotherapy is also used in treating diseases such as autoimmune diseases and viral infections caused by hepatitis, HIV, HPV and Varicella.

Side effects from these treatments include fatigue, nausea, sleep disturbance and the development of widespread chronic pain.

Drug and radiation therapy can damage peripheral nerves and lead to neuropathic pain. In most cases, nerve injury occurs in tandem with damage to other structures and the pain has mixed somatic and neuropathic components. Often the side effects of radiation and chemotherapy limit the use of these drugs for treatment. The most common side effects are bone marrow suppression, neutropenia, renal toxicity and the induction of peripheral neuropathy and often result in termination of treatment or alteration of the dose.

Chemotherapeutic agents used as therapies include 1) alkylating agents such as mechlorethamine, cyclophosphamide, ifosfamide, chlorambucil, chloroethyl diazihydroxide, isocyanate, and platinum agents; 2) antimetabolites such as folate analogs, purine analogs, pyrimidine analogs, adenosine analogs and substituted ureas; 3) antitumor antibiotics such as blenoxane; 4) anthracyclines; 5) epipodophyllotoxins; 6) vinca alkaloids; 7) camptothecin analogs such as CPT-11 and topotecan; and 8) taxanes such as paclitaxel and docetaxel.

Interferons are used to treat some types of cancer and viral infection. There are three major types of interferons—interferon alpha, interferon beta, and interferon gamma; interferon alpha is the type most widely used in cancer treatment. Consensus interferon is another therapy that combines several different types of interferon and is somewhat unique in its activity, but is associated with side effects similar to those seen with other IFNs. These agents stimulate cellular processes to fight the disease. Side effects of interferon therapy include muscle aches, bone pain, headaches, cognitive deficits, fatigue, nausea and vomiting. There is evidence to support the role of interferon therapy in the generation of neuropathic pain (Emir et al Pediatr Hematol Oncol 1999 16:557-560; Quattrini et al Acta Neuropathol 1997 94:504-508). Administration of IFN-α is frequently accompanied by the appearance of neuropsychiatric symptoms such as depressed mood, anhedonia, anxiety, cognition impairment and neurovegetative and somatic symptoms such as anorexia, fatigue, altered sleep, pain and fever. The neuropsychiatric effects of IFN-α generally resolve after treatment but is some cases can persist for months.

There is evidence to suggest that radiation therapy also contributes to the development of chronic pain in patients treated for breast, prostate, head/neck cancer and Hodgkin's lymphoma (Tasmuth et al 1997 Acta Oncol 36(6):625-30; McFarlane et al. 2002 Clin Oncol 14(6):468-471; Antolak et al 2002 J Urol 167(6):2525; Ehrnrooth et al 2001 Acta Oncol 40:745-750).

The type of resulting neuropathy can be dependent on the type of therapeutic substance used. Platinum derivatives such as cisplatin, oxaliplatin and carboplatin result in a pure sensory and painful neuropathy while substances like vinscristine, taxol and suramin cause a mixed sensorimotor neuropathy with or without involvement of the autonomic nervous system. Peripheral neuropathy resulting from cisplatin dosing is usually not apparent until a cumulative dose of at least 200-350 mg/m² has been administered (Cavaletti et al Cancer 1992 69:203-207; LoMonoco et al J Neurol 1992 239:199-204, Thompson et al Cancer 1984 54:1269-1275). Symptoms of peripheral neuropathy usually appear during the course of therapy, although they can worsen or first develop several months after discontinuing treatment.

The neurotoxicity caused by the chemotherapy is dependant on the total cumulative dose, duration of treatment and type of substance used. Neurotoxicity can occur immediately during or shortly after drug administration. Neurotoxic effects can also become evident a long time after the end of the treatment. In general, the peripheral nervous system is capable of regeneration after injury if the cell body is spared and no further damage occurs during the repair period. However, in some situations, chemotherapy-induced neuropathy is only partly reversible or completely irreversible.

Risk factors have been identified which may predispose an individual to developing neuropathy after chemotherapy. These include familial history (i.e. inherited neuropathy), alcohol use and abuse, and previous nerve damage by diabetes (Zuk et al Folia Neuropathol 2001 39:281-284; Rowinsky et al Semin Oncol 1993 20(4 suppl 3):1-15; Quasthoff and Hartung J Neurol 2002 249:9-17).

An illustrative example is Post Breast Surgery Pain Syndrome (PBSPS) which is an underreported condition believed to affect 10-30% of women who have undergone surgical treatment for breast cancer. It is now believed that radiation and chemotherapy play a role in aggravating the condition (Lash and Silliman J Clin Epidemial 2000; 53:615-622). PBSPS is primarily a neuropathic disorder believed to be caused by a number of factors including injury to nerves/tissue during surgery, radiation therapy or chemotherapy. Chemotherapy using agents such as Taxol, Vincristine, and Platinum) can contribute to polyneuropathies similar to those induced by radiation therapy and thus intensify the pain and impairment caused by surgery. Symptoms can include chest and upper arm pain, numbness, edema, continuous aching and burning associated with chronic dysesthesia, allodynia and phantom breast tactile sensation/pain.

II. Compositions

A. Non-Tricyclic Reuptake Inhibitors.

In a preferred embodiment a monoamine reuptake inhibitor is administered to treat chronic pain associated with drug or radiation therapy. These compounds are capable of blocking reuptake of NE, 5-HT or DA or combinations thereof. In a more preferred embodiment, an NSRI is administered to treat chronic pain associated with drug or radiation therapy by blocking reuptake of NE or 5-HT. In the most preferred embodiment, the NSRI is milnacipran.

This compound is preferably administered in an effective amount to alleviate the symptoms of chronic pain associated with drug or radiation therapy.

Monoamine reuptake inhibitors are known in the art and function by blocking transport proteins that selectively re-sequester the monoamine back into the axon terminal. For example, dopamine reuptake inhibitory activity typically involves blocking the dopamine transporter (DAT) such that dopamine reuptake is inhibited. The ability of a compound to block the DAT or increase release of dopamine can be determined using several techniques known in the art. For example, Gainetdinov et al., (1999, Science, 283: 397-401), describes a technique in which the extracellular dopamine concentration in the striatum can be measured using microdialysis. The extracellular concentration of dopamine can be measured before and after administration of the compound to determine the ability of a compound to block the DAT or increase the release of dopamine. A statistically significant increase in dopamine levels post-administration of the compound being tested indicates that the compound inhibits the reuptake of dopamine or increases the release of dopamine. The ability to block the DAT can also be quantified with inhibitory concentration (IC) values, like IC₅₀, at the dopamine transporter. Several techniques for determining IC values are described in the art. (For example, see Rothman et al., 2000, Synapse, 35:222-227) These techniques can be applied for NE and 5-HT as well. The compounds useful in these methods typically have IC₅₀ values in the range of 0.1 nM to 600 μM. In particular, the compounds have IC₅₀ values of 0.1 nM to 100 μM.

TRI compounds, which inhibit the reuptake of serotonin, noradrenaline, and dopamine, can be used. A specific example of a TRI compound is sibutramine (BTS 54 524; N-[1-[1-(4-chlorophenyl)cyclobutyl]-3-methylbutyl]-N,N-dimethylamine hydrochloride monohydrate), or a pharmaceutically acceptable salt thereof. Sibutramine blocks the reuptake of the neurotransmitters dopamine, norepinephrine, and serotonin. The chemical structure of sibutramine is well known in the art. This compound is described in U.S. Pat. No. 4,939,175 and Buckett et al.,(Prog. Nuero-Psychopharmacol. & Biol. Psychiat 1988 vol. 12:575-584).

In a preferred embodiment, the DRI compounds are NSRI compounds and exhibit a greater inhibition of norepinephrine reuptake than serotonin reuptake. In one embodiment, the NSRI compounds have a ratio of inhibition of norepinephrine reuptake to serotonin reuptake (“NE:5-HT”) of about 2-60:1, i.e., the NSRI compound is about 2-60 times better at inhibiting reuptake of norepinephrine compared to inhibiting reuptake of serotonin. NE>5-HT SNRI compounds having a NE:5-HT ratio of about 10:1 to about 2:1 are thought to be particularly effective.

Various techniques are known in the art to determine the NE:5-HT of a particular SNRI. For example, the ratio can be calculated from IC₅₀ data for NE and 5-HT reuptake inhibition. It has been reported that for milnacipran the IC₅₀ of norepinephrine reuptake is 100 nM, whereas the IC₅₀ of serotonin reuptake inhibition is 200 nM. See Moret et al., (Neuropharmacology, 24(12):1211-1219, 1985); Palmier, C, et al. (1989). Therefore, the NE:5-HT reuptake inhibition ratio for milnacipran based on this data is 2:1. Of course, other IC values such as IC₂₅, IC₇₅, etc. could be used, so long as the same IC value is being compared for both norepinephrine and serotonin. The concentrations necessary to achieve the desired degree of inhibition (i.e., IC value) can be calculated using known techniques either in vivo or in vitro. See Sanchez and Hyttel (Cell Mol Neurobiol 19(4): 467-89); Turcotte et al (Neuropsychopharmacology. 2001 May;24(5):511-21); Moret et al. (Neuropharmacology 1985 Dec;24(12):1211-9.); Moret and Briley (Neuropharmacology. 1988 Jan;27(1):43-9); Bel and Artigas (Neuropsychopharmacology 1999 Dec;21(6):745-54); Palmier et al (Eur J Clin Pharmacol 1989;37(3):235-8).

Additional SNRI compounds that can be used include aminocyclopropane derivatives disclosed in WO95/22521; U.S. Pat. No. 5,621,142; Shuto et al. J. Med. Chem., 38:2964-2968, 1995; Shuto et al., J. Med. Chem., 39:4844-4852, 1996; Shuto et al., J. Med. Chem., 41:3507-3514, 1998; and Shuto et al., 85:207-213, 2001, that are structurally related to milnacipran and may inhibit the reuptake of norepinephrine more than they inhibit reuptake of serotonin. Using the 2-60 range defined above, one could also use reboxetine and, possibly, atomoxetine.

Milnacipran and methods for its synthesis are described in U.S. Pat. No. 4,478,836. Additional information regarding milnacipran may be found in the Merck Index, 12th Edition, at entry 6281. Unless specifically noted otherwise, the term “milnacipran” as used herein refers to both enantiomerically pure forms of milnacipran as well as to mixtures of milnacipran enantiomers.

Another SNRI compound is duloxetine, or a pharmaceutically acceptable salt thereof. Duloxetine is usually administered to humans as the hydrochloride salt and most often administered as the (+) enantiomer. The chemical structure of duloxetine is well known to those skilled in the art. Duloxetine and methods for its synthesis are described in U.S. Pat. No. 4,956,388. Additional information regarding duloxetine may be found in the Merck Index, 12th Edition, at entry 3518.

Another specific example of an SNRI compound is venlafaxine, or a pharmaceutically acceptable salt thereof. The chemical structure of venlafaxine is well known to those skilled in the art. Venlafaxine and methods for its synthesis are described in U.S. Pat. Nos. 4,535,186 and 4,761,501. Additional information regarding venlafaxine may be found in the Merck Index, 12th Edition, at entry 10079. It is understood that venlafaxine as used herein refers to venlafaxine's free base, its pharmaceutically acceptable salts, its racemate and its individual enatiomers, and venlafaxine analogs, both as racemates and as their individual enantiomers.

Those of skill in the art will recognize that SNRI compounds such as milnacipran may exhibit the phenomena of tautomerism, conformational isomerism, geometric isomerism and/or optical isomerism. For example, as is clear from the above structural diagram, milnacipran is optically active. It has been reported in the literature that the dextrogyral enantiomer of milnacipran is about twice as active in inhibiting norepinephrine and serotonin reuptake than the racemic mixture, and that the levrogyral enantiomer is much less potent (see, e.g., Spencer and Wilde, 1998, supra; Viazzo et al., 1996, Tetrahedron Lett. 37(26):4519-4522; Deprez et al., 1998, Eur. J. Drug Metab. Pharmacokinet. 23(2): 166-171). Accordingly, milnacipran administered in enantiomerically pure form (e.g., the pure dextrogyral enantiomer) or as a mixture of dextrogyral and levrogyral enantiomers, such as a racemic mixture. Methods for separating and isolating the dextro- and levrogyral enantiomers of milnacipran and other SNRI compounds are well-known (see e.g., Grard et al., 2000, Electrophoresis 2000 21:3028-3034).

It will also be appreciated that in many instances the SNRI compounds may be metabolized to produce active SNRI compounds and that active metabolites could be used.

Glutaminergic neurotransmission plays a key role in the central sensitization that can cause the hypersensitivity sometimes associated with chronic pain. Therefore compounds that inhibit glutaminergic neurotransmission, like NMDA antagonists, can be particularly useful in treating chronic pain associated with drug or radiation therapy. It has been reported that milnacipran and its derivatives have antagonistic properties at the NMDA receptor. See Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. The SNRI compounds with NMDA receptor antagonistic properties can have IC₅₀ values from about 1 nM-100 μM. For example, milnacipran has been reported to have an IC₅₀ value of about 6.3 μM. The NMDA receptor antagonistic properties of milnacipran and its derivatives are described in Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. Methods for determining the antagonism and affinity for antagonism are disclosed in Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213.

Aminocyclopropane derivatives disclosed in WO95/22521; U.S. Pat. No. 5,621,142; Shuto et al., J. Med. Chem., 38:2964-2968, 1995; Shuto et al., J. Med. Chem., 39:4844-4852, 1996; Shuto et al., J. Med. Chem., 41:3507-3514, 1998; and Shuto et al., Jpn. J. Pharmacol., 85:207-213, 2001 that inhibit reuptake of NE more than 5-HT and have NMDA antagonistic properties also can be used.

B. Other Active Agents Administered with DRIs

DRI compounds are effective in treating chronic pain when administered alone (or in combination with other compounds that are not neurotransmitter precursors such as phenylalanine, tyrosine and/or tryptophan). The DRI compounds such as milnacipran, can be administered adjunctively with other active compounds such as antidepressants, analgesics, muscle relaxants, anorectics, stimulants, antiepileptic drugs, and sedative/hypnotics. Specific examples of compounds that can be adjunctively administered with the DRI compounds include, but are not limited to, neurontin, pregabalin, pramipexole, L-DOPA, amphetamine, tizanidine, clonidine, tramadol, morphine, tricyclic antidepressants, codeine, cambamazepine, sibutramine, amphetamine, valium, trazodone and combinations thereof.

Typically, for a patient undergoing drug or radiation therapy, the DRI compound may be adjunctively administered with antidepressants, anorectoics, analgesics, antiepileptic drugs, muscle relaxants, and sedative/hypnotics. Adjunctive administration, as used herein, means simultaneous administration of the compounds, in the same dosage form, simultaneous administration in separate dosage forms, and separate administration of the compounds. For example, milnacipran can be simultaneously administered with valium, wherein both milnacipran and valium are formulated together in the same tablet. Alternatively, milnacipran can be simultaneously administered with valium, wherein both the milnacipran and valium are present in two separate tablets. In another alternative, milnacipran can be administered first followed by the administration of valium, or vice versa. These compounds would preferably be administered in an effective amount to alleviate widespread chronic pain associated with drug or chemotherapy.

III. Methods of Treatment

The compounds can be administered therapeutically to achieve a therapeutic benefit or prophylactically to achieve a prophylactic benefit. By therapeutic benefit is meant eradication or amelioration of the underlying disorder being treated, e.g., eradication or amelioration of the chronic pain associated with drug or radiation therapy, and/or eradication or amelioration of one or more of the symptoms associated with the underlying disorder such that the patient reports an improvement in feeling or condition, notwithstanding that the patient may still be afflicted with the underlying disorder. For example, administration of milnacipran to a patient suffering from chronic pain provides therapeutic benefit not only when the underlying chronic pain is eradicated or ameliorated, but also when the patient reports decreased symptoms of the chronic pain in the patient, for example, decreased fatigue, improvements in sleep patterns, and/or a decrease in the severity or duration of pain.

For therapeutic administration, the compound typically will be administered to a patient already diagnosed with the particular indication being treated.

For prophylactic administration, the compound may be administered to a patient prior to receiving drug or radiation therapy, or to a patient reporting one or more of the physiological symptoms of chronic pain, even though a diagnosis attributing it to drug or radiation therapy may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. For example, the compound could be prophylactically administered prior to bedtime to avoid the sleep disturbances associated with chronic pain. Alternatively, the compound could be administered prior to recurrence or onset of a particular symptom, for example, pain, or fatigue.

The compounds, or pharmaceutically acceptable salts thereof, can be formulated as pharmaceutical compositions, including their polymorphic variations. Such compositions can be administered orally, buccally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The term “pharmaceutically acceptable salt” means those salts which retain the biological effectiveness and properties of the compounds used in the present invention, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that other carboxylic acid derivatives, for example carboxylic acid amides, including carboxamides, lower alkyl carboxamides, di(lower alkyl) carboxamides, could be used.

The compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

The compounds may be complexed with other agents. The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); or lubricants. If any such formulated complex is water-soluble, then it may be formulated in an appropriate buffer, for example, phosphate buffered saline or other physiologically compatible solutions. Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration.

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

The compounds may also be formulated in rectal compositions such as suppositories or retention enemas, e.g., containing conventional suppository bases such as cocoa butter or other glycerides. Suppositories for rectal or vaginal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal or vaginal temperature, and which will therefore melt in the rectum or vagina and release the drug.

Solid dosage forms for oral administration may include capsules, tablets, pills, powders, and granules. In such solid dosage forms, the compounds of this invention are ordinarily combined with one or more adjuvants appropriate to the indicated route of administration. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

Alternatively, for oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid) and sweetening, flavoring, and perfuming agents.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The amount of active ingredient that can be combined with the carrier materials to produce a single dosage form will vary depending upon the patient and the particular mode of administration. Preparations for oral administration may be suitably formulated to give controlled release of the active compound.

For administration by inhalation, the compounds may be delivered in the form of an aerosol spray or dry powder inhaler.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active compound(s) may be in powder form for constitution with a suitable vehicle, e.g., sterile pyrogen-free water, before use.

In addition to the formulations described previously, the compounds may also be formulated as a depot or sustained-release preparation. Such long acting formulations may be administered by implantation, osmotic pump or transcutaneous delivery (for example subcutaneously or intramuscularly), intramuscular injection or a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

b. Effective Dosages

Therapeutically effective amounts for use in humans can be determined from animal models. For example, a dose for humans can be formulated to achieve circulating concentration that has been found to be effective in animals. Useful animal models for these syndromes are known in the art.

Effective amounts for use in humans can be also be determined from human data for the compounds used to treat depression. The amount administered can be the same amount administered to treat depression or can be an amount lower than the amount administered to treat depression. Doses for oral administration of a DRI compound typically range from about 1 μg-1 gm/day. For example, the amount of milnacipran administered to prevent depression is in the range of about 50 mg-100 mg/day. For the treatment of chronic pain, the dosage range for milnacipran is typically from 25 mg-400 mg/day, more typically from 100 mg-250 mg/day. The dosage may be administered once per day or several or multiple times per day. The amount of the compound will be dependent on the subject being treated, the severity of the affliction, the manner of administration and the judgment of the prescribing physician.

1. A method of treating chronic widespread pain associated with drug or radiation therapy comprising administering to a patient undergoing or having recently undergone drug or radiation therapy, an effective amount of a pharmaceutical compound selected from the group consisting of dual reuptake inhibitor (DRI) pharmaceutical compounds and triple reuptake inhibitor (TRI) pharmaceutical compounds, to alleviate chronic widespread pain associated with the drug or radiation therapy.

2. The method of claim 1 wherein the DRI is an SNRI compound.

3. The method of claim 1 wherein the DRI is an NSRI compound.

4. The method of claim 1 wherein the DRI compound has NMDA antagonist activity.

5. The method of claim 3 wherein the NSRI compound has NMDA antagonist activity.

6. The method of claim 2 wherein the SNRI compound is selected from the group consisting of duloxetine and venlafaxine.

7. The method of claim 3 wherein the NSRI compound is milnacipran.

8. The method of claim 1 wherein the TRI compound has NMDA antagonist activity.

9. The method of claim 1 wherein the TRI is sibutramine.

10. The method of claim 7, wherein the amount administered is from about 25 mg to about 400 mg per day.

11. The method of claim 10 wherein the amount administered is from approximately 100 mg per day to 250 mg per day.

12. The method according to claim 1, wherein the compound is formulated in a sustained release dosage formulation.

13. The method of claim 1 wherein the disease is selected from the group consisting of cancer, viral infection, rheumatoid arthritis and autoimmune disease.

14. The method of claim 12 wherein the disease is cancer.

15. The method of claim 1 wherein the patient is undergoing radiation therapy.

16. The method of claim 1 wherein the patient is undergoing chemotherapy.

17. The method of claim 1 wherein the patient recently had radiation therapy.

18. The method of claim 1 wherein the patient recently had chemotherapy.

19. The method of claim 1 wherein the drug is administered just before chemotherapy or radiation.
